(19) United States
(12) Patent Application Publication
(10) Pub. No.: US 2026/0041485 A1
(43) Pub. Date: Feb. 12, 2026

(54) APPARATUS AND METHODS ASSOCIATED WITH AN ELECTROSURGICAL WIRE

(71) Applicant: ElectroWire Corporation, Lexington, SC (US)

(72) Inventors: Robert Leonardi, Lexington, SC (US); Peter J. D'Aquanni, Murriega, CA (US)

(21) Appl. No.: 19/274,873

(22) Filed: Jul. 21, 2025

Related U.S. Application Data
(60) Provisional application No. 63/680,719, filed on Aug. 8, 2024.
(60) Provisional application No. 63/758,058, filed on Feb. 13, 2025.

(51) Int. Cl. A61B 18/14; A61B 18/00

(52) U.S. Cl. CPC A61B 18/1492; A61B 2018/00083; A61B 2018/00357

(57) ABSTRACT

An apparatus for performing a medical procedure including an electrosurgical unit for generating radiofrequency energy, an interventional wire including a distal tip portion configured to deliver the radiofrequency energy to tissue during the medical procedure and an activator to activate delivery of the radiofrequency energy from the electrosurgical unit to the interventional wire. The distal tip portion of the interventional wire includes a core wire in a tubular structure. A distal tip structure or element at the end of the core wire delivers the radiofrequency energy to tissue.

### CROSS REFERENCE TO RELATED APPLICATIONS

This application claims the benefit of U.S. Provisional Patent Application Ser. No. 63/680,719 filed Aug. 8, 2024 (pending), and U.S. Provisional Patent Application Ser. No. 63/758,058, filed Feb. 13, 2025 (pending), the disclosures of which are incorporated by reference herein in their entirety.

### TECHNICAL FIELD

The present disclosure generally relates to medical equipment and methods using electrosurgical wires and, particularly, wires having distal tips for applying radiofrequency energy to tissue.

### BACKGROUND

Advances in interventional medicine have caused an increase in various minimally invasive procedures such as transseptal and other endovascular procedures. Transseptal procedures known as transseptal punctures are critical to gain access to the heart. More specifically, transseptal punctures allow medical professionals to gain access to the left atrium of the heart. Access to the left atrium of the heart is obtained through the right atrium of the heart after the medical professional has gained access to the heart, such as either from the jugular vein through the superior vena cava, or from the femoral vein through the inferior vena cava.

Conventional transseptal procedures utilize needles to make the puncture across the septum from the right atrium of the heart to the left atrium of the heart to allow the medical professional to gain access to the left atrium. Needles, though, are not the most ideal manner of making this type of puncture. Needles tend to slide when advanced with the intent to puncture, leading to off-target punctures and further, needles cannot be utilized to deliver necessary medical devices to the left atrium, so additional steps are required when these medical devices need to access the left atrium.

Radiofrequency-assisted devices are also being used to puncture through septal tissue within the heart and/or to gain access to the left atrium of the heart. Some such technologies use a radiofrequency energy-based guidewires to pierce the septum located between the right atrium of the heart and the left atrium of the heart. But radiofrequency-assisted guidewire devices of this type can be expensive and, therefore, this technology may be limited in use. For example, existing radiofrequency-assisted devices used for septal heart tissue puncturing may require expensive electrosurgical, RF energy generating units, as opposed to the more conventional, less costly electrosurgical, RF energy generating units used for many surgical procedures other than septal puncturing of heart tissue. Also, existing radiofrequency-assisted devices used for septal heart tissue puncturing may be incompatible with preferred transseptal introducer sets, may require connection to electrosurgical pencils for operation, and may not be stiff enough to optimally support delivery of bulky interventional devices.

For these and other reasons, there is a continuing need in this field for radiofrequency-assisted devices, systems and methods that may be used to perform medical procedures, such as transseptal and other endovascular procedures.

### SUMMARY

An apparatus is provided for performing a medical procedure and generally includes an electrosurgical unit for generating radiofrequency energy (RF), an interventional wire including a distal tip portion configured to deliver the radiofrequency energy to tissue during the medical procedure and an activator electrically connecting the electrosurgical unit and the interventional wire to activate delivery of the radiofrequency energy from the electrosurgical unit to the interventional wire. The distal tip portion of the interventional wire includes a core wire positioned in a first tube, a second tube positioned around the first tube and an electrically insulative layer surrounding and in contact with the first tube while leaving a distal tip of the interventional wire exposed for delivering the RF energy to target tissue. The tubes may be solid metallic tubes or solid non-metallic tubes, such as hypotubes, or one or both tubes may have a different construction such as a coil structure and/or one or both tubes may be formed from other materials such as other electrically conductive materials or electrically non-conductive materials, for example. At least the core wire conducts the RF energy to the distal tip for purposes of the medical procedure. If one or both tubes are formed from electrically conductive material(s), then one or both tubes may also conduct RF energy. In this case, for example, the tube or tubes may conduct the RF energy to the distal tip and the core wire may not need to extend all the way to the distal tip.

The distal tip portion of the interventional wire may further include an electrically conductive distal tip element. The distal tip element may contact the distal ends of the first and second tubes for delivery of the RF energy. The electrically conductive distal tip element may further comprise a weld. The distal tip portion of the interventional wire may further include a coil structure positioned between the first tube and the electrically insulative layer. Optionally, the first tube may comprise a coil structure.

A method of performing a medical procedure is provided and includes generating radiofrequency (RF) energy, conducting the radiofrequency energy through an elongated, flexible conductive element, selectively directing the radiofrequency energy from the elongated, flexible conductive element to a distal tip portion of an interventional wire and delivering the radiofrequency energy to tissue through a structure at the distal tip portion of the interventional wire. The structure includes a core wire positioned in a first tube, a second tube positioned around the first tube and an electrically insulative layer surrounding and in contact with a portion of the first tube while leaving a distal tip exposed to deliver the RF energy to the tissue.

The structure at the distal tip portion of the interventional wire may further include an electrically conductive distal tip element contacting the distal ends of the first and second tubes. The radiofrequency energy may be delivered through the electrically conductive distal tip element to the tissue. The electrically conductive distal tip element may be a weld and the radiofrequency energy may, in that case, be delivered through the weld. The distal tip portion of the interventional wire may further include a coil structure positioned between the first tube and the electrically insulative layer. Optionally, as mentioned herein as one of various alternatives or additional features, the first tube may be constructed as a coil structure. The medical procedure performed may be a transseptal puncture. Radiofrequency energy may be delivered to a proximal end of the elongated, flexible conductive element. Radiofrequency energy may be delivered through a distal end of the elongated, flexible conductive element to the interventional wire. Radiofrequency energy may be delivered through a wire or cable, for example, that acts as at least a part of the elongated, flexible conductive element.

An alternative apparatus is also provided for performing a medical procedure including an electrosurgical unit for generating radiofrequency energy, an interventional wire including a distal tip portion configured to deliver the radiofrequency energy to tissue during the medical procedure and an activator electrically connecting the electrosurgical unit and the interventional wire to activate delivery of the radiofrequency energy from the electrosurgical unit to the interventional wire. The distal tip portion of the interventional wire includes a core wire positioned in a tubular structure the tubular structure including a first portion and a second portion. The second portion is positioned more distal than the first portion and may be directly adjacent the first portion or not. The second portion has a larger outer diameter than the first portion and may also have a larger outer diameter than an immediately adjacent and more distal third portion. In this case the second portion is a projecting portion that projects radially from immediately adjacent more proximal and more distal portions, respectively. In this case as well, a step may exist that provides a “step down” in diameter between the second and third portions may serve as a retainer for the distal end portion or, more specifically, a terminus of an electrically insulative layer. The electrically insulative layer surrounds the first portion of the tubular structure. In one illustrative embodiment, the tube may have one or more “steps” that create abruptly changing diameters. In other embodiments, adjacent diameters of the tube may change more gradually such as with desired radii or tapers that prevent sharp corners at the transitions between changing diameters. The core wire preferably has a constant diameter extending through the distal tip portion. The tubular structure may be formed of one or more separate tubes having different outer diameters, or may have one or more integrally formed sections of different outer diameters. In this latter case, for example, the integrally formed sections could be machined or otherwise manufactured from or into a single, solid part formed of one or more materials.

The electrically insulative layer may contact the first portion of the tubular structure. The distal tip portion of the interventional wire may further include an electrically conductive distal tip element contacting a distal end of the tubular structure. The electrically conductive distal tip element may further include a weld. The distal tip portion of the interventional wire may further include a coil structure positioned between the first portion of the tubular structure and the electrically insulative layer. The elongated, flexible conductive element may include a distal end, and the distal end is electrically coupled to the interventional wire. The elongated, flexible conductive element may be, for example, a wire or cable.

The apparatus may further comprise a coil structure positioned between the electrically insulative layer and the first portion of the tubular structure. The first portion of the tubular structure may further comprise a non-constant outer diameter along a length thereof assisting with retention of the coil structure on the first portion of the tubular structure. The first portion of the tubular structure may alternatively or also comprise a non-constant outer diameter along a length thereof assisting with retention of the electrically insulative layer on the first portion of the tubular structure. A third portion of the tubular structure may be positioned adjacent and distal to the second portion of the tubular structure. The third portion of the tubular structure may comprise a non-constant diameter along a length thereof. A fourth portion of the tubular structure may be positioned adjacent to and distal to the third portion of the tubular structure. The fourth portion of the tubular structure may have a larger diameter along a length thereof than the third portion of the tubular structure. The fourth portion of the tubular structure may further include a proximal surface, such as an abrupt step, providing a stop for a distal end of the electrically insulative layer. The tubular structure may comprise a stepped tubular structure having abrupt changes in diameter between adjacent first, second, third and fourth portions thereof or as otherwise contemplated herein.

An alternative method of performing a medical procedure is provided and includes generating radiofrequency energy, conducting the radiofrequency energy through an elongated, flexible conductive element, selectively directing the radiofrequency energy from the elongated, flexible conductive element to a distal tip portion of an interventional wire and delivering the radiofrequency energy to tissue through a structure at the distal tip portion of the interventional wire. The structure includes a core wire positioned in a tubular structure, the tubular structure including a first portion and a second portion. The second portion is positioned more distal than the first portion and has a larger outer diameter along a length thereof than the first portion. An electrically insulative layer surrounds the first portion. As mentioned above, the tubular structure may have one or more “steps” that create abruptly changing diameters. In other embodiments, adjacent diameters of the tubular structure may change more gradually such as with desired radii or tapers that prevent sharp corners at the transitions between changing diameters. The core wire preferably has a constant diameter extending through the distal tip portion. The tubular structure may be formed of one or more separate tubes having different outer diameters, or may have one or more integrally formed sections of different outer diameters as contemplated herein.

The distal tip portion of the interventional wire may further include a coil structure positioned between the proximal portion of the tubular structure and the electrically insulative layer. The medical procedure performed may be a transseptal puncture. Radiofrequency energy may be delivered to a proximal end of the elongated, flexible conductive element. Radiofrequency energy may be delivered through a distal end of the elongated, flexible conductive element to the interventional wire. Radiofrequency energy may be delivered through a wire or cable, for example, that acts as the elongated, flexible conductive element.

In various embodiments, the tubular structure may include a projecting portion such as described above and/or hereinbelow, the projecting portion having an outer diameter that is larger than immediately adjacent more proximal and more distal portions of the tubular structure. The projecting portion may be formed with an abruptly changing diameter (i.e., in a “stepped” manner) or in another manner, such as with a more radiused or tapered change in diameter than a typical “step” so as to avoid sharp corners at the transitions between changing diameters. The projecting portion may be integrally formed in the tubular structure or may be a separate tubular element that is suitably secured to another tube. The step up or increase in diameter at the proximal end of the projecting portion may be further configured to accommodate and stop a coil structure, which may be positioned between the tubular structure and the electrically insulative layer. The coil structure may be secured to the stepped tubular structure by welding or any other manner. The step down or decrease in diameter at the distal end of the projecting portion may establish an adjacent third portion lying between the projecting and one or more distal portions of the tubular structure. The third portion may be slightly larger or smaller in diameter than the portion of the tubular structure lying proximal to the projecting portion. The step down or decrease in diameter at the distal end of the projecting portion may be further configured to accommodate and retain a distal terminus of the electrically insulative layer. The variable diameters of the tubular structure may help to maintain a smooth outer contour, especially in conjunction with the electrically insulative layer. Alternatively, such as in a coil-free design, the entirety of a proximal portion of the tubular structure may be larger in diameter than a central portion located between proximal and distal portions of the tubular structure.

In another illustrative embodiment, an apparatus for performing a medical procedure includes an electrosurgical unit for generating radiofrequency energy, and an interventional wire including a distal tip portion configured to deliver the radiofrequency energy to tissue during the medical procedure. An activator is electrically connected to the electrosurgical unit and the interventional wire to activate delivery of the radiofrequency energy from the electrosurgical unit to the interventional wire. The distal tip portion of the interventional wire includes (i) a core wire positioned in a tubular structure, the tubular structure including a first portion and a second portion, the second portion positioned more distal than the first portion and having a larger outer diameter along a length thereof than the first portion, and (ii) an electrically insulative layer surrounding the first portion and further extending at least partially along the more distal second portion of the tubular structure.

The electrically insulative layer may contact the first portion of the tubular structure. The distal tip portion of the interventional wire may further comprise an electrically conductive distal tip element contacting a distal end of the tubular structure and/or the core wire. The electrically conductive distal tip element may further comprise a weld. The distal tip portion of the interventional wire may further comprise a coil structure positioned between the tubular structure and the electrically insulative layer. The first and second portions may be formed integrally with each other or may be separate components affixed together in a suitable manner. The distal tip portion of the interventional wire may further include a coil structure positioned between the tubular structure and the electrically insulative layer. The first portion of the tubular structure may enable fixation of the coil structure to the tubular structure, and the second portion of the tubular structure may act as a distal stop for the coil structure. A coil structure may be positioned between the electrically insulative layer and the first portion of the tubular structure. The first portion of the tubular structure may further comprise a non-constant outer diameter along a length thereof assisting with retention of the coil structure on the first portion of the tubular structure. The first portion of the tubular structure may alternatively or additionally further comprise a non-constant outer diameter along a length thereof assisting with retention of the electrically insulative layer on the first portion of the tubular structure. A third portion of the tubular structure may be positioned adjacent and distal to the second portion of the tubular structure, the third portion of the tubular structure optionally comprising a non-constant diameter along a length thereof for purposes of assisting with retaining the electrically insulative layer. A fourth portion of the tubular structure positioned adjacent to and distal to the third portion of the tubular structure may also be provided. The fourth portion of the tubular structure may have a larger diameter along a length thereof than the third portion of the tubular structure. The fourth portion of the tubular structure may also have a proximal edge providing a stop for a distal end of the electrically insulative layer. The tubular structure may comprise a stepped tubular structure having changes in diameter between adjacent first, second, third and fourth portions thereof such as discussed herein.

In another aspect, a method of performing a medical procedure includes generating radiofrequency energy, conducting the radiofrequency energy through an elongated, flexible conductive element, and selectively directing the radiofrequency energy from the elongated, flexible conductive element to a distal tip portion of an interventional wire. The radiofrequency energy is delivered to tissue through a structure at the distal tip portion of the interventional wire. The structure includes (i) a core wire positioned in a tubular structure, the tubular structure including a first portion and a second portion, the second portion positioned distal to the first portion, and the first portion having a larger outer diameter along a length thereof than the more distal second portion, and (ii) an electrically insulative layer surrounding the first portion of the tubular structure and further extending at least partially along the second portion of the tubular structure. The tubular structure may also include a projecting portion as previously described, and may otherwise be formed with one or more features such as those described herein.

Various additional features and advantages will become readily apparent to those of ordinary skill in the art upon review of the following detailed description of the illustrative embodiments, taken in conjunction with the accompanying drawings.

### BRIEF DESCRIPTION OF THE DRAWINGS

FIG. 1 is a perspective view of an illustrative apparatus for delivering RF energy to tissue during a medical procedure.

FIG. 2 is an enlarged plan view of a distal tip portion of the interventional wire that is part of the apparatus shown in FIG. 1.

FIG. 2A is a longitudinal cross-sectional view of the distal tip portion shown in FIG. 2.

FIG. 3 is a plan view of a pigtail loop formed by the interventional wire of the apparatus.

FIG. 4 is an enlarged plan view of an alternative distal tip portion of the interventional wire that is part of the apparatus shown in FIG. 1.

FIG. 4A is a longitudinal cross-sectional view of the distal tip portion shown in FIG. 4.

FIG. 5 is an enlarged plan view of another alternative distal tip portion of the interventional wire that is part of the apparatus shown in FIG. 1.

FIG. 5A is a longitudinal cross-sectional view of the distal tip portion shown in FIG. 5.

FIG. 6 is an enlarged plan view of another alternative distal tip portion of the interventional wire that is part of the apparatus shown in FIG. 1.

FIG. 6A is a longitudinal cross-sectional view of the distal tip portion shown in FIG. 6.

### DETAILED DESCRIPTION

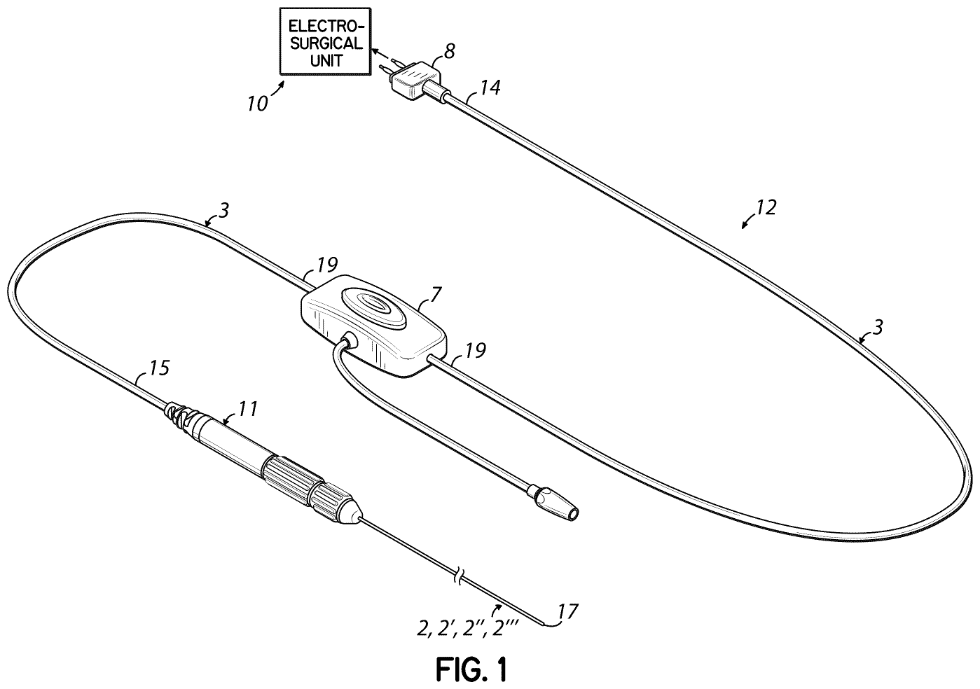

FIG. 1 is a perspective view of an illustrative apparatus for delivering RF energy to tissue during a medical procedure. Like reference numerals are used to refer to like elements and function as between the different embodiments herein, while like reference numerals having prime marks (′), double prime marks (″) and triple prime marks (″′) refer to like elements of the first, second, third, and fourth embodiments (FIGS. 1 through 6A) but having slightly different structure and/or function which will be either apparent to those of ordinary skill in the art and/or further described herein below. In this illustrative embodiment, an interventional wire 2, an elongated flexible conductive element 3, an activator unit 7, an electrosurgical unit 10, and a coupler 11 form the apparatus 12 which is configured for performing a medical procedure. It will be appreciated that many other forms of the apparatus may be used in carrying out the invention as set forth in one or more claims. The examples given herein are illustrative in nature and in no way limit the scope of the invention. The elongated flexible conductive element 3 is an electrically insulated conductor, such as an electrically insulated cable or wire, configured for transmitting electricity or RF energy. In some embodiments, the elongated flexible conductive element 3 may be a cable including an insulated wire or wires and having a protective casing. The elongated flexible conductive element 3 includes a proximal end 14 including an electrosurgical unit connector 8, and a distal end 15 coupled to the coupler 11. A distal tip 17 applies the RF energy to tissue. This distal tip 17 may be an element, such as described below. Various additional exemplary features of the illustrative apparatus shown in FIG. 1 are shown and described in U.S. patent application Ser. No. 18/243,927, the disclosure of which is incorporated by reference herein without limitation on the present invention. As discussed generally above, the embodiments described in illustrative fashion herein pertain to a distal tip portion and multiple illustrative examples of a distal tip portion are shown in the drawing figures and described in the description to follow. It will be appreciated that further embodiments (examples) may be practiced while still embodying one or more inventive principles.

Still referring to FIG. 1, the electrosurgical unit connector 8, capable of attaching to conventional RF energy generating units for delivering RF energy, may releasably connect to the electrosurgical unit 10. Many commercially available electrosurgical units include a standardized receptacle, such as a monopolar accessory receptacle. The electrosurgical unit connector 8 may be configured to couple the apparatus 12 to any one of a plurality of electrosurgical units with standardized receptacles. Therefore, the subsystem or assembly comprising, for example, interventional wire 2, elongated flexible conductive element 3, activator unit 7, and coupler 11 may be physically coupled to one of several different standardized receptacles of an electrosurgical RF generating unit. In the illustrative embodiment, elongated flexible conductive element 3 has a mid-portion 19 that is connected to the activator unit 7. The activator unit 7 may be situated at a location spatially separated from the proximal end 14 of the elongated flexible conductive element 3 and, therefore also spatially separated from the electrosurgical unit 10, by a segment of the elongated flexible conductive element 3. The activator unit 7 also may be spatially separated from the coupler 11 by another segment of the elongated flexible element 3, as shown in FIG. 1 or, optionally, the activator unit 7 may be integrated with or otherwise fixed to the coupler 11. In some embodiments, the activator unit 7 may be located at the proximal end 14 of the elongated flexible conductive element 3 adjacent to or otherwise fixed to or part of the electrosurgical unit connector 8. The elongated flexible conductive element 3 may be constructed in many ways, such as in multiple, discrete pieces or segments or the segments referred to herein may be portions of the same integral, elongated flexible conductive element 3.

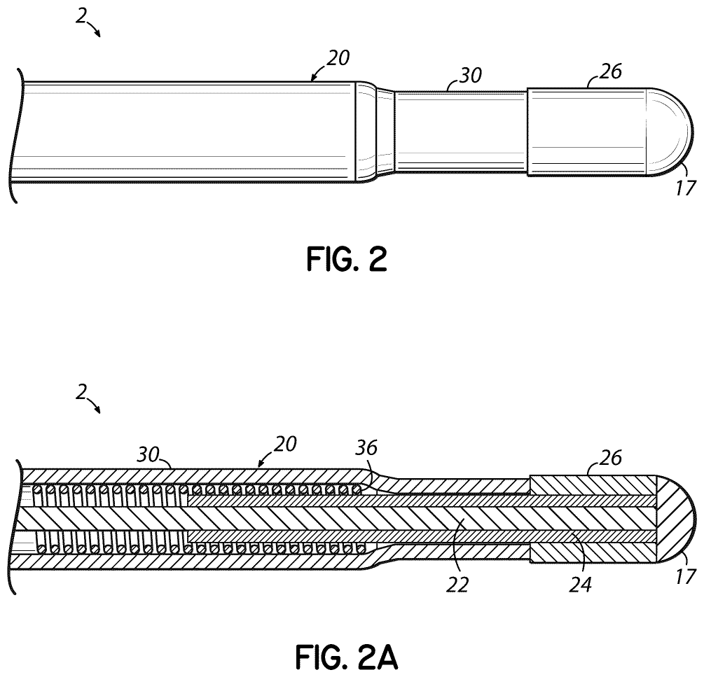

As further detailed in FIGS. 2 and 2A, the interventional wire 2 includes a distal tip portion 20 terminating in the distal tip element 17 which is used to apply the RF energy to tissue during an interventional or endovascular procedure such as, but not limited to, a septal puncture. The distal tip portion 20 of the interventional wire 2 includes an elongate member (core wire) 22 (FIG. 2A) positioned in, such as by being surrounded by and in contact with a first tube 24, a second tube 26 surrounding and in contact with the first tube 24 and an electrically insulative tube 30 or another form of electrical insulation layer material surrounding and in contact with a proximal portion of the first tube 24. The first and second tubes 24, 26 may be, for example, hypotubes, or otherwise may be formed of suitable material, depending on the particular design and function preferences, such as one or more electrically conductive metals. Preferably, when metal is chosen as the material of construction, the chosen metals will have high heat and electrical conductivity and will be easy to weld both to each other and to the core wire 22. One such metal is a biocompatible stainless steel although other metals and/or alloys are also possible. Optionally, one or both tubes 24, 26 may be formed of material(s) that are non-conductive. In this latter case, for example, the core wire 22 would extend to the distal tip element 17 or otherwise be configured with one or more other conductive elements so as to conduct RF energy to the distal tip element 17.

The distal tip element 17 of the interventional wire 2 is electrically conductive and electrically contacts the core wire 22 and/or the distal ends of the first and second tubes 24, 26 in the illustrated embodiment. The electrically conductive distal tip element 17 may further comprise a weld. The distal tip portion 20 of the interventional wire 2 may further include a coil structure 36 (FIG. 2A) positioned generally between the first tube 24 and the electrically insulative layer 30. A distal end portion of the coil 36 surrounds a proximal end portion of the first tube 24. The electrically insulative layer 30 surrounds the coil 36 and the first tube 24 in this embodiment.

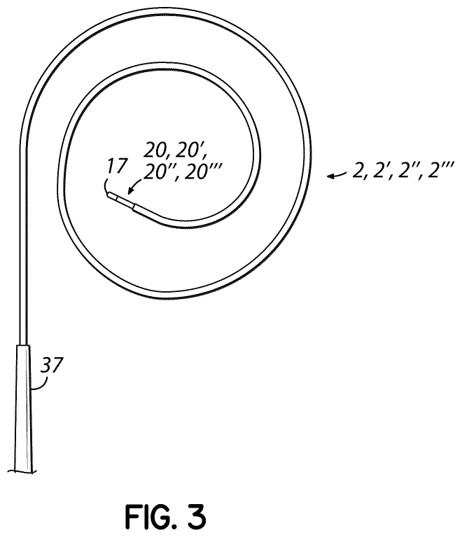

A small diameter of the core wire 22 allows adequate flexibility of the distal pigtail shape of the interventional wire 2 (FIG. 3). The nested tube design of the distal tip portion 20 increases stiffness, allowing push forces to be transmitted to the distal tip element 17 without guidewire deformation and/or fracture as the distal tip portion 20 is advanced beyond the tip of a dilator 37 of an introducer set. Combined with RF energy, for example, the stiffness of the distal tip portion 20 facilitates successful inter-atrial septal puncture without the need for a sharpened distal tip element 17. The nested mass of the first and second tubes 24, 26 over the core wire 22 also serves as a heat sink, limiting heating of the core wire 22 and decreasing risk of heat-related fracture of the core wire 22 with RF energy application. The nested mass of the first and second tubes 24, 26 over the core wire 22 also serves to improve radiopacity (and, therefore, fluoroscopic visibility) of the distal tip portion 20, which may be further improved through incorporation of highly radiopaque alloys.

The nested mass of the first and second tubes 24, 26 over the core wire 22 is also easily assembled during manufacturing. The first tube 24 has an outer diameter that is slightly smaller than the inner diameter of the coil 36, facilitating welding of the coil 36 to the outer surface of the first tube 24. The second tube 26 has an inner diameter that is slightly larger than the outer diameter of the first tube 24, which has an inner diameter that is slightly larger than the outer diameter of the core wire 22. The core wire 22 is allowed to pass entirely through the aligned distal ends of the first and second tubes 24, 26 during manufacturing. The protruding distal tip of the core wire 22 can be melted (such as by plasma welding) to create the rounded distal tip element 17 that permanently bonds the first and second tubes 24, 26 to each other and to the core wire 22.

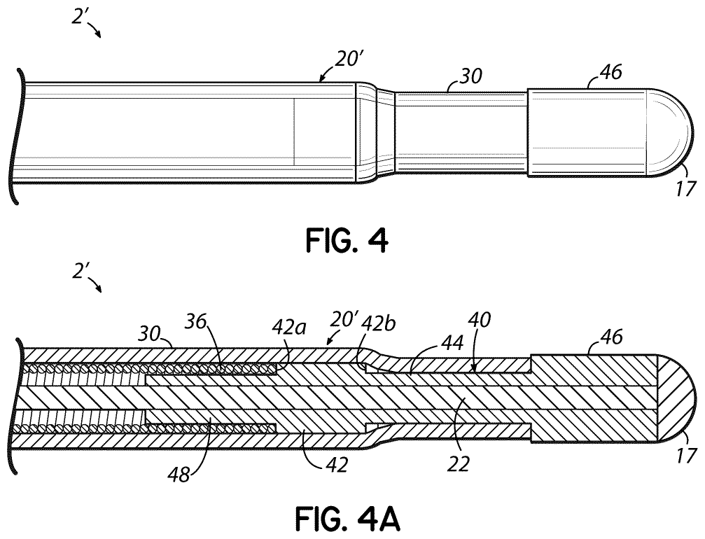

Referring to FIGS. 4 and 4A, a first alternative and illustrative embodiment is shown. In this embodiment, like reference numerals are used to refer to like elements and function as between the different embodiments, while like reference numerals having prime marks (′) refer to like elements of the first embodiment but having slightly different structure and/or function which will be either apparent to those of ordinary skill in the art and/or described herein. Further description of such like elements is not given hereinbelow so as to reduce redundancy, except as necessary or desirable in describing this illustrative embodiment. The interventional wire 2′ includes a distal tip portion 20′ terminating in the distal tip element 17. As with the first embodiment, the distal tip element 17 is used to apply the RF energy to tissue during an interventional or endovascular procedure such as, but not limited to, a septal puncture. As more specifically illustrated in the cross-sectional view of FIG. 4A, the distal tip portion 20′ of the interventional wire 2′ includes a core wire 22 surrounded by and in contact with a stepped tube 40 having a proximal portion 48, a projecting portion 42 which projects in a radially outward direction relative to the proximal portion 48 as well as relative to a central or more distal portion 44. Finally, this embodiment of the stepped tube further includes a distal tip portion 46. These portions of tube 40 (or any other embodiment of a tubular structure as contemplated herein) may be respectively considered first, second, third and fourth portions. One or more of the portions may be removed and additional portions may be added. The portions may be formed integrally with one another or as an assembly of separate tubes of various diameters secured into a cohesive structure. It will be understood that the various portions of the tube 40 may have portions thereof, such as one or more of portions 42, 44, 46, 48 formed with a continuous tubular design or with one or more individual, radially extending protruding areas and/or recessed areas that create a non-constant outer diameter and serve desired functions such as those described herein. The previously described electrically insulative tube 30 or another form of electrical insulation layer material surrounds and may be in contact with any conductive portions of the proximal portion 48 (which may be wholly or partially covered by the coil 36), radially projecting portion 42, and more distal portion 44 of the stepped tube 40. The insulative layer (such as tube 30) does not extend to cover the distal tip portion 46 of the stepped tube 40 or the rounded distal tip element 17 in this embodiment. Portion 42 may have a constant or variable (non-constant or non-uniform) diameter at one or more areas thereof that is larger than portions 44 and 48 of the stepped tube 40. In any event, portion 42 may have a greater diameter than the portions immediately adjacent to it, such that a coil structure 36 (proximally) and the distal terminus of an electrically insulative layer 30 (distally) may be accommodated and retained while maintaining a smooth outer contour. Alternatively, such as in a coil-free design, the entirety of portion 48 of the stepped tube 40 may be larger in diameter than portion 44 located between portions 46 and 42, such that the distal terminus of electrically insulative layer 30 may be accommodated and retained while maintaining a smooth outer contour.

As shown in the alternative embodiment of FIGS. 4 and 4A, and generally described above, the portions 42, 44, 46, 48 of the stepped tube 40 (FIG. 4A) may have abruptly variable (i.e., changing) diameters, which may be established by assembly of parts and/or from a single substrate or material. For example, the relatively larger portions 42 and/or 46 of the stepped tube 40 may be established by fixation of an outer hypotube or hypotubes to an inner hypotube. Fixation may be with a weld or by another suitable method. Alternatively, the relatively smaller proximal portions 48 and/or 44 of the stepped tube 40 may be established by removal of material from a single substrate or material (e.g., a tube), such as by computer numerical control or “CNC” machining, centerless grinding, subtractive manufacturing or another method. Furthermore, any of the portions 42, 44, 46, 48 of the stepped tube 40 may be manufactured with a material, such as one or more metals, that conduct electricity and heat. Preferably, the chosen metals will have high heat and electrical conductivity and will be easy to weld both to each other and to the core wire 22. One such metal is a biocompatible stainless steel although other metals and/or alloys are also possible. In other embodiments, the tube 40 may be formed from one or more non-conductive materials or even both conductive and non-conductive materials.

The distal tip element 17 of the interventional wire 2′ is electrically conductive and electrically contacts a distal end of the stepped tube 40 in the illustrative embodiment. The electrically conductive distal tip element 17 may further include a weld. The distal tip portion 20′ of the interventional wire 2′ may further include a coil structure 36 (FIG. 4A) positioned generally between the stepped tube 40 and the electrically insulative layer 30. The coil 36 may extend proximally over the core wire 22 for a desired distance (not shown) for reasons understood by those of ordinary skill in the art. The coil 36 may extend distally over portion 48 of the stepped tube 40, where the coil 36 may be welded and/or otherwise secured to portion 48 of the stepped tube 40. A proximal end 42a of portion 42 may serve as a stop for the coil 36. As further detailed below and shown in FIG. 4A, portion 42 also serves to retain the electrically insulative tube 30, allowing push forces to be transmitted to the distal tip element 17 without deformation and/or proximal sliding of the electrically insulative layer 30 as the distal tip portion 20′ of the interventional wire 2′ is advanced beyond the tip of the dilator 37 of the introducer set (FIG. 3), for example, during an inter-atrial septal puncture or other procedure.

The stepped tube 40 is easily assembled over the core wire 22 during manufacturing. Portion 42 of the stepped tube 40 may have an outer diameter similar in size to the outer diameter of the coil 36, and portion 44 of the stepped tube 40 may have an outer diameter slightly smaller than the outer diameter of the coil 36. Thus, the proximal end or edge 42a of portion 42 can serve as a stop for the distal end of the coil 36, and a distal end or edge 42b of portion 42 can serve as a step down over which the distal terminus of the electrically insulative layer 30 is directed (such as by a shrink manufacturing process) to help retain the distal terminus of the insulative layer 30 in position on the stepped tube 40. The distal portion of the coil 36 may extend over (around) portion 48 of the stepped tube 40, enabling fixation of the coil 36 to portion 48 of the stepped tube 40 by welding or another fixation method. The abruptly variable diameters of the portions 42, 44, 46, 48 of the stepped tube 40 can keep the outer contour of the distal tip portion 20′ of the interventional wire 2′ relatively smooth across the distal termini of the coil 36 and the electrically insulative layer 30. The core wire 22 is allowed to pass entirely through the distal end of the stepped tube 40 during manufacturing. The protruding distal tip of the core wire 22 can be melted (such as by plasma welding) to create the rounded distal tip element 17 that permanently bonds the distal end of the stepped tube 40 to the core wire 22.

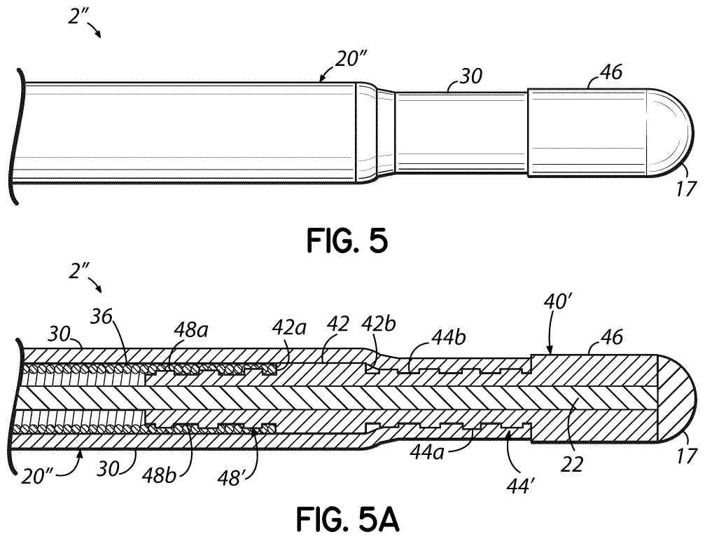

Referring to FIGS. 5 and 5A, another alternative, illustrative embodiment is shown. In this embodiment, like reference numerals are used to refer to like elements and function as between the different embodiments, while like reference numerals having prime marks (′) or double prime marks (″) refer to like elements of the first and/or second embodiments (FIGS. 1 through 4A) but having slightly different structure and/or function which will be either apparent to those of ordinary skill in the art or described herein. Further description of such like elements may not be given hereinbelow so as to reduce redundancy, except as necessary or desirable in describing this illustrative embodiment. The embodiment of the stepped tube shown in FIGS. 5 and 5A is similar to the embodiment described immediately above (FIGS. 4 and 4A), except that stepped tube portions 44′ and 48′ have an illustrative embodiment or type of non-constant outer diameter. It will be appreciated that each of the distal tip embodiments described or contemplated herein may be used with the overall system shown and described in connection with the first embodiment. In this third embodiment of the stepped tube 40′, non-constant outer diameters are formed by respective raised sections or areas 44a, 48a and respective recessed sections or areas 44b, 48b of the distal tip or stepped tube 40′. These raised and recessed areas 44a, 48a, 44b, 48b may be formed, for example, by at least one generally circumferentially extending groove which result(s) in respective raised and recessed sections or areas. This, for example, may be a single generally helical or spiral groove, or a series of successive circular and slightly spaced apart circumferential grooves. Other options (not shown) can be roughening the surface such as through sufficient knurling or other material removal, addition or deformation. In each case, a non-constant outer diameter will be formed on the outer surface(s) of tube portion 44′ and/or tube portion 48′ sufficient to result in greater retention ability of coil 36 on portion 48′ and/or greater retention ability of insulative layer or material 30 on portion 44′. While the non-constant diameter(s) of portions 44′ and/or 48′ are designed to assist with retention, the larger diameter tube portion 42 can remain in this embodiment to: a) add a more prominent distal edge that can provide even more effective retention of the electric insulation layer 30 as described in connection with FIG. 4A, and/or b) add a “hard stop” at a proximal edge as a manufacturing aid in properly and consistently locating the distal position of the coil 36 during assembly as also shown and described in connection with FIG. 4A. Other aspects of the illustrative embodiment shown in FIGS. 5 and 5A may be understood as described above in connection with the description of FIGS. 4 and 4A taken together with FIGS. 1, 2, 2A and 3.

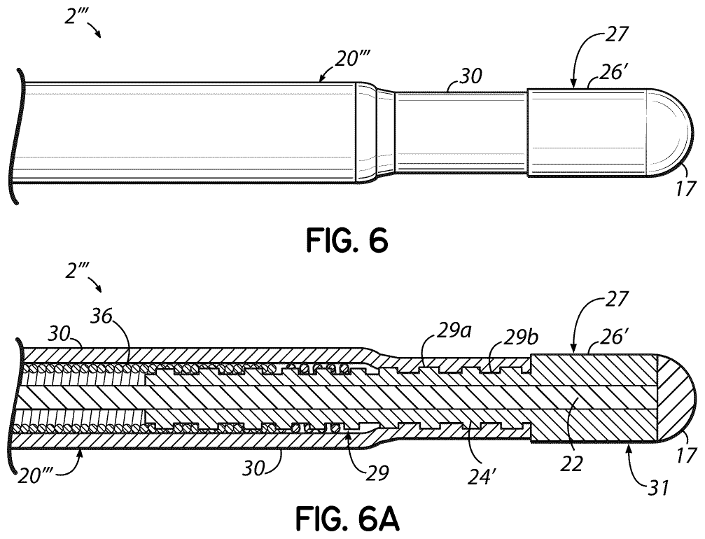

Referring to FIGS. 6 and 6A, another alternative, illustrative embodiment is shown. In this embodiment, like reference numerals are used to refer to like elements and function as between the different embodiments, while like reference numerals having prime marks (′), double prime marks (″) and triple prime marks (″′) refer to like elements of the first, second and third embodiments (FIGS. 1 through 5A) but having slightly different structure and/or function which will be either apparent to those of ordinary skill in the art or described herein. Further description of such like elements may not be given hereinbelow so as to reduce redundancy, except as necessary or desirable in describing this illustrative embodiment. The tip construction which comprises the embodiment of FIGS. 6 and 6A is similar to the tip construction shown in the first embodiment described above (FIGS. 2 and 2A), with two primary differences. One is that the two tubes 24, 26 shown in FIG. 2A have been integrated into a single tube 27 with a proximal portion 29 and a distal portion 31 in this alternative embodiment. The proximal portion 29 of tube 27 has a non-constant outer diameter. As with the previous embodiment, the non-constant outer diameter may be formed by raised sections or areas 29a and recessed sections or areas 29b. These raised and recessed areas 29a, 29b may be formed, for example, by at least one generally circumferentially extending groove which result(s) in respective raised and recessed sections or areas. This, for example, may be a single generally helical or spiral groove, or a series of successive circular, circumferential grooves. Other options can be roughening the surface such as through sufficient knurling or other material removal, addition or deformation. In each case, a non-constant outer diameter will be formed on proximal tube portion 29 sufficient to result in greater retention ability of coil 36 on portion 29 and/or greater retention ability of insulative layer or material 30 on portion 29. Other aspects of the illustrative embodiment shown in FIGS. 6 and 6A may be understood as described above in connection with the description of FIGS. 1 through 5A.

While the present invention has been illustrated by the description of specific embodiments thereof, and while the embodiments have been described in considerable detail, it is not intended to restrict or in any way limit the scope of the appended claims to such detail. The various features discussed herein may be used alone or in any combination within and between the various embodiments. Additional advantages and modifications will readily appear to those skilled in the art. For example, the first tube 24 might be replaced by a variation of coil 36 or by an additional coil. Or, both tubes 24, 26 might be replaced by a single tube with a machined outer profile similar to the outer diameter profile of the nested mass of the two tubes 24, 26 previously described. As another example, any number of the portions forming the stepped tube 40 or other embodiment of a tubular structure may be made by assembly of parts or from a single material or substrate. As a further example, the illustrative “portions” of the tubular structure provided as examples herein may be formed, dimensioned and/or positioned relative to one another in any advantageous manner desired to achieve purposes such as, but not limited to, those exemplified herein. Many other modifications may be made and/or features added or deleted while practicing one or more inventive principles disclosed herein. The invention in its broader aspects is therefore not limited to the specific details, representative apparatus and methods and illustrative examples shown and described. Accordingly, departures may be made from such details without departing from the scope or spirit of the general inventive concepts.

What is claimed is:

1. An apparatus for performing a medical procedure comprising:
an electrosurgical unit for generating radiofrequency energy;
an interventional wire including a distal tip portion configured to deliver the radiofrequency energy to tissue during the medical procedure; and
an activator electrically connecting the electrosurgical unit and the interventional wire to activate delivery of the radiofrequency energy from the electrosurgical unit to the interventional wire;
wherein the distal tip portion of the interventional wire includes:
i. a core wire positioned in a first tube,
ii. a second tube positioned around the first tube; and
iii. an electrically insulative layer surrounding and in contact with a proximal portion of the first tube.

2. The apparatus of claim 1, wherein the distal tip portion of the interventional wire further comprises an electrically conductive distal tip element in electrical contact with the core wire.

3. The apparatus of claim 2, wherein the electrically conductive distal tip element further comprises a weld and fixes distal ends of the first and second tubes together.

4. The apparatus of claim 2, wherein the distal tip portion of the interventional wire further comprises a coil structure positioned between the first tube and the electrically insulative layer.

5. A method of performing a medical procedure comprising:
generating radiofrequency energy;
conducting the radiofrequency energy through an elongated, flexible conductive element;
selectively directing the radiofrequency energy from the elongated, flexible conductive element to a distal tip portion of an interventional wire; and
delivering the radiofrequency energy to tissue through a structure at the distal tip portion of the interventional wire, the structure including:
i. a core wire positioned in a first tube,
ii. a second tube positioned around the first tube; and
iii. an electrically insulative layer surrounding and in contact with a proximal portion of the first tube.

6. The method of claim 5, wherein the structure at the distal tip portion of the interventional wire further comprises an electrically conductive distal tip element and the radiofrequency energy is delivered through the electrically conductive distal tip element to the tissue.

7. The method of claim 6, wherein the electrically conductive distal tip element further comprises a weld and the radiofrequency energy is delivered through the weld.

8. The method of claim 5, wherein the distal tip portion of the interventional wire further comprises a coil structure positioned between the first tube and the electrically insulative layer.

9. The method of claim 5, wherein the medical procedure performed is a transseptal puncture.

10. The method of claim 5, wherein radiofrequency energy is delivered to a proximal end of the elongated, flexible conductive element.

11. The method of claim 5, wherein radiofrequency energy is delivered through a distal end of the elongated, flexible conductive element to the interventional wire.

12. The method of claim 5, wherein radiofrequency energy is delivered through a wire that acts as the elongated, flexible conductive element.

13. The method of claim 5, wherein radiofrequency energy is delivered through a cable that acts as the elongated, flexible conductive element.

14. An apparatus for performing a medical procedure comprising:
an electrosurgical unit for generating radiofrequency energy;
an interventional wire including a distal tip portion configured to deliver the radiofrequency energy to tissue during the medical procedure; and
an activator electrically connecting the electrosurgical unit and the interventional wire to activate delivery of the radiofrequency energy from the electrosurgical unit to the interventional wire;
wherein the distal tip portion of the interventional wire includes:
i. a core wire positioned in a tubular structure, the tubular structure including a first portion and a second portion, the second portion positioned more distal than the first portion, the second portion having a larger outer diameter along a length thereof than the first portion; and
ii. an electrically insulative layer surrounding the first portion of the tubular structure.

15. The apparatus of claim 14, wherein the electrically insulative layer contacts the first portion of the tubular structure.

16. The apparatus of claim 14, wherein the distal tip portion of the interventional wire further comprises an electrically conductive distal tip element contacting a distal end of the tubular structure and/or the core wire.

17. The apparatus of claim 16, wherein the electrically conductive distal tip element further comprises a weld.

18. The apparatus of claim 16, wherein the distal tip portion of the interventional wire further comprises a coil structure positioned between the tubular structure and the electrically insulative layer.

19. The apparatus of claim 14, wherein the first and second portions are formed integrally with each other.

20. The apparatus of claim 14, wherein the distal tip portion of the interventional wire further comprises a coil structure positioned between the tubular structure and the electrically insulative layer, the first portion of the tubular structure enables fixation of the coil structure to the tubular structure, and the second portion of the tubular structure acts as a distal stop for the coil structure.

21. The apparatus of claim 14, wherein the tubular structure further comprises a third portion positioned more distal than the second portion, and the second portion is a radially projecting portion with an outer diameter of greater dimension than the respective outer diameters of the first and third portions to thereby form a step between the radially projecting portion and the third portion, wherein the electrically insulative layer surrounds the radially projecting portion and extends distally past the step between the radially projecting portion and the third portion so as to retain a distal portion of the electrically insulative layer on the tubular structure.

22. The apparatus of claim 14, further comprising a coil structure positioned between the electrically insulative layer and the first portion of the tubular structure, wherein the first portion of the tubular structure further comprises a non-constant outer diameter along a length thereof assisting with retention of the coil structure on the first portion of the tubular structure.

23. The apparatus of claim 14, wherein the first portion of the tubular structure further comprises a non-constant outer diameter along a length thereof assisting with retention of the electrically insulative layer on the first portion of the tubular structure.

24. The apparatus of claim 23, further comprising a third portion of the tubular structure positioned adjacent and distal to the second portion of the tubular structure, the third portion of the tubular structure comprising a non-constant diameter along a length thereof.

25. The apparatus of claim 24, further comprising a fourth portion of the tubular structure positioned adjacent to and distal to the third portion of the tubular structure, the fourth portion of the tubular structure having a larger diameter along a length thereof than the third portion of the tubular structure.

26. The apparatus of claim 25, wherein the fourth portion of the tubular structure further comprises a proximal edge providing a stop for a distal end of the electrically insulative layer.

27. The apparatus of claim 26, wherein the tubular structure comprises a stepped tubular structure having abrupt changes in diameter between adjacent first, second, third and fourth portions thereof.

28. A method of performing a medical procedure comprising:
generating radiofrequency energy;
conducting the radiofrequency energy through an elongated, flexible conductive element;
selectively directing the radiofrequency energy from the elongated, flexible conductive element to a distal tip portion of an interventional wire; and
delivering the radiofrequency energy to tissue through a structure at the distal tip portion of the interventional wire, the structure including:
i. a core wire positioned in a tubular structure, the tubular structure including a first portion and a second portion, the second portion positioned more distal than the first portion, the second portion having a larger outer diameter along a length thereof than the first portion; and
ii. an electrically insulative layer surrounding the first portion of the tubular structure.

29. The method of claim 28, wherein the structure at the distal tip portion of the interventional wire further comprises an electrically conductive distal tip element contacting a distal end of the tubular structure and/or the core wire, and the radiofrequency energy is delivered through the electrically conductive distal tip element to the tissue.

30. The method of claim 29 wherein the electrically conductive distal tip element further comprises a weld and the radiofrequency energy is delivered through the weld.

31. The method of claim 28, wherein the distal tip portion of the interventional wire further comprises a coil structure positioned between the tubular structure and the electrically insulative layer.

32. The method of claim 28, wherein the medical procedure performed is a transseptal puncture.

33. The method of claim 28, wherein radiofrequency energy is delivered to a proximal end of the elongated, flexible conductive element.

34. The method of claim 28, wherein radiofrequency energy is delivered through a distal end of the elongated, flexible conductive element to the interventional wire.

35. The method of claim 28, wherein radiofrequency energy is delivered through a wire that acts as the elongated, flexible conductive element.

36. The method of claim 28, wherein radiofrequency energy is delivered through a cable that acts as the elongated, flexible conductive element.

37. An apparatus for performing a medical procedure comprising:
an electrosurgical unit for generating radiofrequency energy;
an interventional wire including a distal tip portion configured to deliver the radiofrequency energy to tissue during the medical procedure; and
an activator electrically connecting the electrosurgical unit and the interventional wire to activate delivery of the radiofrequency energy from the electrosurgical unit to the interventional wire;
wherein the distal tip portion of the interventional wire includes:
i. a core wire positioned in a tubular structure, the tubular structure including a first portion and a second portion, the second portion positioned more distal than the first portion and having a larger outer diameter along a length thereof than the first portion; and
ii. an electrically insulative layer surrounding the first portion and further extending at least partially along the more distal second portion of the tubular structure.

38. The apparatus of claim 37, wherein the electrically insulative layer contacts the first portion of the tubular structure.

39. The apparatus of claim 37, wherein the distal tip portion of the interventional wire further comprises an electrically conductive distal tip element contacting a distal end of the tubular structure and/or the core wire.

40. The apparatus of claim 39, wherein the electrically conductive distal tip element further comprises a weld.

41. The apparatus of claim 39, wherein the distal tip portion of the interventional wire further comprises a coil structure positioned between the tubular structure and the electrically insulative layer.

42. The apparatus of claim 37, wherein the first and second portions are formed integrally with each other.

43. The apparatus of claim 37, wherein the distal tip portion of the interventional wire further comprises a coil structure positioned between the tubular structure and the electrically insulative layer, the first portion of the tubular structure enables fixation of the coil structure to the tubular structure, and the second portion of the tubular structure acts as a distal stop for the coil structure.

44. The apparatus of claim 37, wherein the tubular structure further comprises a third portion positioned more distal than the second portion, and the second portion is a radially projecting portion with an outer diameter of greater dimension than the respective outer diameters of the first and third portions to thereby form a step between the radially projecting portion and the third portion, wherein the electrically insulative layer surrounds the radially projecting portion and extends distally past the step between the radially projecting portion and the third portion so as to retain a distal portion of the electrically insulative layer on the tubular structure.

45. The apparatus of claim 37, further comprising a coil structure positioned between the electrically insulative layer and the first portion of the tubular structure, wherein the first portion of the tubular structure further comprises a non-constant outer diameter along a length thereof assisting with retention of the coil structure on the first portion of the tubular structure.

46. The apparatus of claim 37, wherein the first portion of the tubular structure further comprises a non-constant outer diameter along a length thereof assisting with retention of the electrically insulative layer on the first portion of the tubular structure.

47. The apparatus of claim 46, further comprising a third portion of the tubular structure positioned adjacent and distal to the second portion of the tubular structure, the third portion of the tubular structure comprising a non-constant diameter along a length thereof.

48. The apparatus of claim 47, further comprising a fourth portion of the tubular structure positioned adjacent to and distal to the third portion of the tubular structure, the fourth portion of the tubular structure having a larger diameter along a length thereof than the third portion of the tubular structure.

49. The apparatus of claim 48, wherein the fourth portion of the tubular structure further comprises a proximal edge providing a stop for a distal end of the electrically insulative layer.

50. The apparatus of claim 48, wherein the tubular structure comprises a stepped tubular structure having abrupt changes in diameter between adjacent first, second, third and fourth portions thereof.

51. A method of performing a medical procedure comprising:
generating radiofrequency energy;
conducting the radiofrequency energy through an elongated, flexible conductive element;
selectively directing the radiofrequency energy from the elongated, flexible conductive element to a distal tip portion of an interventional wire; and
delivering the radiofrequency energy to tissue through a structure at the distal tip portion of the interventional wire, the structure including:
i. a core wire positioned in a tubular structure, the tubular structure including a first portion and a second portion, the second portion being positioned distal to the first portion and the first portion having a larger outer diameter along a length thereof than the more distal second portion; and
ii. an electrically insulative layer surrounding the first portion and further extending at least partially along the second portion of the tubular structure.
